(19) United States
(12) Patent Application Publication
(10) Pub. No.: US 2024/0252322 A1
(43) Pub. Date: Aug. 1, 2024

(54) ARTIFICIAL KNEE JOINT REPLACEMENT

(71) Applicants: Kyoung Tak KANG, Seoul (KR); Ji Hoon NAM, Seoul (KR); TJC Life Co., Ltd., Seoul (KR)

(72) Inventors: Yong Gon KOH, Seoul (KR); Kyoung Tak KANG, Seoul (KR); Ji Hoon NAM, Seoul (KR)

(73) Assignees: Kyoung Tak KANG, Seoul (KR); Ji Hoon NAM, Seoul (KR); TJC Life Co., Ltd., Seoul (KR)

(21) Appl. No.: 18/315,845

(22) Filed: May 11, 2023

(30) Foreign Application Priority Data
Jan. 30, 2023 (KR) 10-2023-0011554

(51) Int. Cl. A61F 2/38

(52) U.S. Cl. CPC A61F 2/3868; A61F 2002/30604; A61F 2310/0097

(57) ABSTRACT

Proposed is an artificial knee joint replacement. The artificial knee joint replacement includes a tibial component directly fixed to a bone resection surface of tibia and configured to have a protrusion portion protruding from an upper fastening surface thereof, and a bearing component interposed between a femoral component directly fixed to a bone resection surface of femur and the tibial component, and configured to have an accommodation portion formed on a lower fastening surface thereof to correspond to the protrusion portion, wherein the protrusion portion consists of two protrusions spaced apart from each other along a first direction reference line of the tibial component, and the accommodation portion consists of grooves formed in a structure that is continuous with each other along a first direction reference line of the bearing component.

### CROSS REFERENCE TO RELATED APPLICATION

The present application claims priority to Korean Patent Application No. 10-2023-0011554, filed Jan. 30, 2023, the entire contents of which is incorporated herein for all purposes by this reference.

### BACKGROUND OF THE INVENTION

### Field of the Invention

The present disclosure relates to an artificial knee joint replacement and, more particularly, to an artificial knee joint replacement inserted between the femur and tibia to act as an artificial joint.

### Description of the Related Art

The knee joint refers to a joint made up of three bones that surround the knee: the femur (also called “thigh bone”), the tibia (also called “shin bone”), and the patella (also called “kneecap”). Knee joints are present in both left and right legs, and together with the hip joint (coxa), correspond to the most essential joint enabling humans to walk upright.

The knee joint is frequently used due to its nature, so there is a lot of room for damage. In particular, wear and aging of bone tissue may lead to deterioration or loss of function, and gradual damage to cartilage or damage to bones and ligaments due to degenerative changes may cause inflammation and pain. This is called degenerative arthritis.

To treat degenerative arthritis, conservative treatment such as lifestyle changes, medication, and physical therapy is generally used. Yet, these treatment options only slow down the progression of degenerative arthritis or temporarily relieve pain, but are not a fundamental solution. In fact, there are cases in which degenerative arthritis becomes extremely aggravated and can no longer be treated with medication or physical therapy.

Total knee arthroplasty is performed for patients who continue to have pain despite medication or physical therapy and have difficulty walking due to extremely aggravated degenerative arthritis. Total knee arthroplasty is a surgical procedure in which an articular surface of a severely worn joint is excised and replaced with an artificial knee joint, and the artificial knee joint used in this case is typically composed of a femoral component, a tibial component, and a bearing component.

The femoral component is implanted in the place where the articular surface of the severely worn femur joint is resected, and the tibial component is implanted in the place where the articular surface of the upper end of the tibia adjacent to the femoral joint is resected. The bearing component is positioned between the femoral component and the tibial component and serves as a kind of cartilage so that the articular surface can move smoothly.

FIG. 1A is a schematic view showing a knee joint before and after total knee arthroplasty of a target patient, and FIG. 1B is a drawing substitute photo showing a conventional artificial knee joint used in total knee arthroplasty.

Referring to FIGS. 1A and 1B, an artificial knee joint 10 may consist of a femoral component 11 directly fixed to the femur; a tibial component 13 directly fixed to the tibia; and a bearing component 12 interposed between the femoral component 11 and the tibial component 13 and functioning as a bearing. The bearing component 12 shown in FIGS. 1A and 1B corresponds to a fixed bearing that is coupled to the tibial component 13 for fixation.

The fixed bearing component 12 may be classified into a hook type, a pin type, a dovetail type, etc. depending on a locking mechanism with the tibial component 13. The hook type is better than other types in terms of ease of fastening, and the pin type has an advantage over other types in terms of ease of separation. The dovetail type has an advantage of good fastening stability and easy separation.

The locking mechanism by type will be described in detail. In the case of the hook type, hooks are constructed in a structure that oppose each other at the front and rear ends of the upper surface of the tibial component, and a concave portion in the form in which the hooks are engaged and coupled to is provided on the coupling surface of the bearing component facing the tibial component in order to combine the tibial component and the bearing component.

Although the hook-type locking mechanism makes it easy to fasten the tibial component and the bearing component, when trying to take out the rear hook part in a state where the front and rear hooks are combined, the front hook gets caught on the principle of leverage and it is very difficult to take it out. There is also a problem of poor post-fastening stability on the rear hook side in the combined state.

The pin type is basically similar to the aforementioned hook type. The difference is that, as shown in FIG. 2, a concave portion is formed toward the front side between a pair of hooks 13-1 provided at the front end of the upper surface of a tibial component 13, and in a state where a hook 12-1 provided in the front of the coupling surface of the bearing component is seated in the concave portion, a separate pin 13-2 that functions as a latch is used to couple the bearing component with the tibial component.

Since the pin type is a method of combining the tibial component and the bearing component using a pin so that they are not separated, it is easier to take out the bearing component than in the case of the hook type because only the pin needs to be removed when separating the tibial component and the bearing component. However, there is a structural problem that an additional pin inserted in the lateral direction is required.

As shown in FIGS. 3A and 3B, the dovetail type has better post-fastening stability than the hook type or pin type described above, and it is easy to separate the tibial component and the bearing component since separation is done by inserting a tool into a separation groove 12-6. Still, as shown in FIG. 4C, in the case of the dovetail type, a coupling surface 12-7 has to slide to a designated coupling position while maintaining contact with a separation preventing step 13-5 in the process of fitting a fastening portion 12-3 to an accommodation portion 13-3, which means that a lot of force is required to combine tibial component and the bearing component.

Accordingly, in an actual surgical procedure, an impactor 14 as shown in FIG. 4D or FIG. 5A or a trigger-type dedicated tool 15 or hammer as shown in FIG. 5B is used. The problem is that when an impactor or hammer is used, the impact may cause cracks in the patient's bones, and trigger-type tools require a great grip force to operate, which is inconvenient to use.

For reference, among the directional terms mentioned above, the “front” refers to a direction adjacent to the patella, and means an anterior direction of the body, whereas the “rear” refers to the opposite side of the patella, and means a posterior direction of the body.

The above-mentioned various types of conventional locking mechanisms (hook type, pin type, and dovetail type) are methods in which fastening is performed on the basis of the outline of the tibia during surgery. Thus, when applied to a custom fit total knee replacement, locations of hooks, pinholes, fastening portions, stepped portions, and accommodation portions need to be adjusted according to the planned appearance (appearance of tibia), leading to increase in processing cost. A method that does not utilize the outline of the tibia requires a separate pin.

Moreover, although the direction of the load applied to the knee joint in the actual human body during walking is from the rear to the front, most of the conventional artificial knee joints discussed above are designed to counter the load applied from the front to the rear, and thus there is a structural problem that the bearing component may be separated or displaced from the tibial component when a large load beyond the design limit is applied from the rear to the front.

### DOCUMENTS OF RELATED ART

(Patent Document 0001) Korean Patent No. 10-1769125 (Registered Aug. 10, 2017)

### SUMMARY OF THE INVENTION

Accordingly, the present disclosure has been made keeping in mind the above problems occurring in the related art, and the present disclosure is intended to provide an artificial knee joint replacement that compensates for the disadvantages of the conventional dovetail-type locking mechanism (excessive force required during fastening and separation) while maintaining the advantages of the conventional dovetail-type locking mechanism (high fastening stability).

An objective of the present disclosure is to provide an artificial knee joint replacement suitable for patient customization.

An objective of the present disclosure is to provide an artificial knee joint replacement that exerts strong resistance in the direction against the load applied to the knee joint of the actual human body during walking.

In order to achieve the above objective, according to an embodiment of the present disclosure, there is provided an artificial knee joint replacement, including: a tibial component directly fixed to a bone resection surface of tibia and configured to have a protrusion portion protruding from an upper fastening surface thereof, wherein the protrusion portion may consist of two protrusions spaced apart from each other along a first direction reference line of the tibial component, wherein the two protrusions may include: a first protrusion configured to protrude from the upper fastening surface spaced a first distance from a rear indentation portion of the tibial component, and have a dovetail-shaped cross-sectional structure in which a width or cross-sectional area gradually decreases as going downward; and a second protrusion configured to protrude from the upper fastening surface spaced a second distance from an anterior curvature point of the tibial component, and whose surface facing the first protrusion is inclined downward in a direction of the first protrusion.

The first protrusion may have a planar uppermost surface with a largest area whose shape is a rectangle or square, and may be a protrusion having a dovetail-shaped cross-sectional structure in which a cross-sectional area gradually decreases downward, and the second protrusion may have a cross-sectional shape of a right triangle with a surface facing the first protrusion inclined in the direction of the first protrusion, and may be a protrusion having a smaller width w2 than a width w1 of the first protrusion in a second direction orthogonal to the first direction.

According to another embodiment of the present disclosure, there is provided an artificial knee joint replacement, including: a bearing component interposed between a femoral component directly fixed to a bone resection surface of femur and the tibial component described in claim 1, and configured to have an accommodation portion formed on a lower fastening surface thereof to correspond to the protrusion portion of the tibial component, wherein the accommodation portion may consist of grooves formed in a structure that is continuous with each other along a first direction reference line of the bearing component, wherein the grooves may include: a first groove configured to open toward a front of the bearing component; and a second groove formed in succession behind the first groove deeper than the first groove, wherein the second groove may be subdivided into: a binding area provided with a binding rail; and a binding guide area configured to guide a protrusion formed on the tibial component to slide toward the binding area to be bound.

According to still another embodiment of the present disclosure, there is provided an artificial knee joint replacement, including: a tibial component directly fixed to a bone resection surface of tibia and configured to have a protrusion portion protruding from an upper fastening surface thereof; and a bearing component interposed between a femoral component directly fixed to a bone resection surface of femur and the tibial component, and configured to have an accommodation portion formed on a lower fastening surface thereof to correspond to the protrusion portion, wherein the protrusion portion may consist of two protrusions spaced apart from each other along a first direction reference line of the tibial component, and the accommodation portion may consist of grooves formed in a structure that is continuous with each other along a first direction reference line of the bearing component, wherein a straight line distance d1 between an outermost surface of one of the two protrusions in a first direction and an outermost surface of the remaining protrusion in the first direction may be equal to a length 11 of a remaining groove except for a first groove opened toward a front of the bearing component among the grooves in the first direction or greater than the length 11 in the first direction.

The two protrusions may include: a first protrusion configured to protrude from the upper fastening surface spaced a first distance from a rear indentation portion of the tibial component, and have a dovetail-shaped cross-sectional structure in which a width or cross-sectional area gradually decreases as going downward; and a second protrusion configured to protrude from the upper fastening surface spaced a second distance from an anterior curvature point of the tibial component, and whose surface facing the first protrusion is inclined downward in a direction of the first protrusion.

The grooves may include: the first groove formed to a depth lesser than a height of the second protrusion; and a second groove formed in succession behind the first groove deeper than the first groove, wherein the second groove may be subdivided into: a binding area provided with a binding rail in which a part of an outer surface of the first protrusion having the dovetail-shaped cross-sectional structure is engaged and fixed; and a binding guide area configured to guide the first protrusion to slide toward the binding area to be bound.

Preferably, the first protrusion may have a planar uppermost surface with a largest area whose shape is a rectangle or square, and may be a protrusion having the dovetail-shaped cross-sectional structure in which a cross-sectional area gradually decreases downward.

Preferably, the second protrusion may have a cross-sectional shape of a right triangle with a surface facing the first protrusion inclined in the direction of the first protrusion, and may be a protrusion having a smaller width w2 than a width w1 of the first protrusion in a second direction orthogonal to the first direction.

In addition, a moment the first protrusion moved along the binding guide area reaches a predetermined binding position in the binding area, the second protrusion may be caught on a step formed at a boundary between the first groove and the second groove.

In addition, the bearing component may be a composite of one selected from ultra high molecular weight polyethylene (UHMWPE), polyether ether ketone (PEEK), carbon fiber reinforced polymer, and glass fiber reinforced polymer, or a combination of two or more.

At this time, as the UHMWPE, conventional PE (CPE) or highly crosslinked PE (HXLPE) may be used. In some cases, vitamin E may be further included in the UHMWPE.

An artificial knee joint replacement according to an embodiment of the present disclosure adopts a dovetail locking mechanism but has a structure in which large resistance does not occur throughout the locking process unlike the case of the related art, except for a momentary resistance due to physical contact between a second protrusion and a first groove at the final stage of locking.

That is, according to an artificial knee joint replacement according to an embodiment of the present disclosure, it is possible to compensate for the disadvantages of the conventional dovetail-type locking mechanism (excessive force required during fastening and separation) while maintaining the advantages of the dovetail-type locking mechanism (high fastening stability).

In addition, according to an artificial knee joint replacement according to an embodiment of the present disclosure, since protrusion or accommodation portions formed for combining two mutually locked artificial joint components (tibial component and bearing component) are located on the reference line in the first direction at a certain distance from the edge of the outermost surface of the corresponding component, the processing allowance (cutting allowance) is sufficiently secured, and thus it is suitable for patient customization.

Furthermore, an artificial knee joint replacement according to an embodiment of the present disclosure has a structure that can exert strong resistance in the direction against the load applied to the knee joint of the human body during walking unlike the conventional dovetail type, in which the bearing component is locked from the front to the rear of the tibial implant, generating a large resistance in the direction opposite to the direction of the load applied to the knee joint of the actual human body during walking.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1A is a schematic view showing a knee joint before and after total knee arthroplasty of a target patient, and FIG. 1B is a drawing substitute photo showing a conventional artificial knee joint used in total knee arthroplasty;

FIG. 2 is a view showing a pin type locking mechanism among various types of conventional locking mechanisms for locking a bearing component and a tibial component;

FIGS. 3A and 3B are views showing a dovetail type locking mechanism among various types of conventional locking mechanisms for locking the bearing component and the tibial component;

FIGS. 4A to 4E are views showing a process of locking the bearing component and the tibial component by using the dovetail type locking mechanism of the related art;

FIGS. 5A and 5B are views showing a state in which tools are used in the process of locking the bearing component and the tibial component by using the dovetail type locking mechanism of the related art;

FIG. 6 is an exploded perspective view of an artificial knee joint replacement according to an embodiment of the present disclosure;

FIG. 7 is a perspective view of the artificial knee joint replacement shown in FIG. 6 viewed from another angle;

FIG. 8 is a combined cross-sectional view of the artificial knee joint replacement according to an embodiment of the present disclosure;

FIGS. 9A to 9C are views of the tibial component applied to the artificial knee joint replacement according to an embodiment of the present disclosure;

FIGS. 10A to 10C are views of the bearing component applied to the artificial knee joint replacement according to an embodiment of the present disclosure;

FIGS. 11A to 11C are perspective views of a locking process of the artificial knee joint replacement according to an embodiment of the present disclosure; and

FIGS. 12A to 12C are views of the locking process of the artificial knee joint replacement according to an embodiment of the present disclosure viewed from the right side of the artificial knee joint replacement.

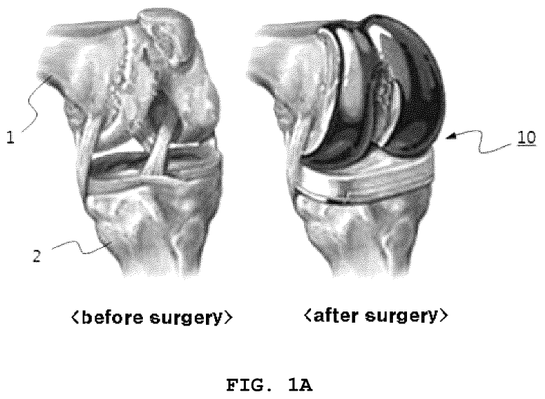

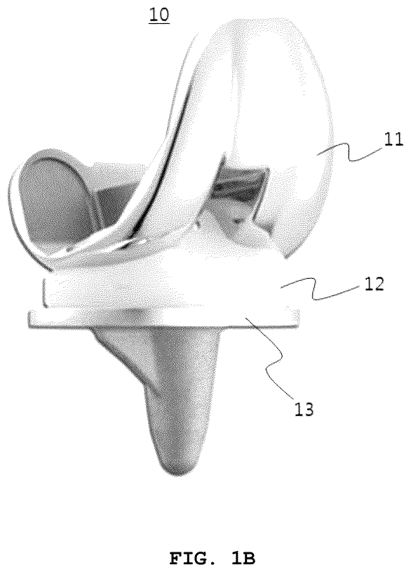

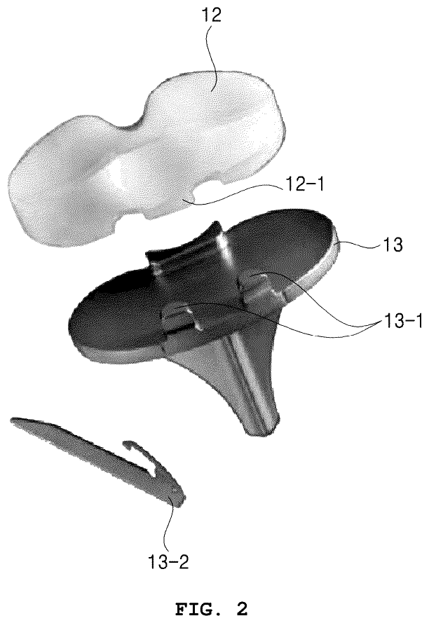

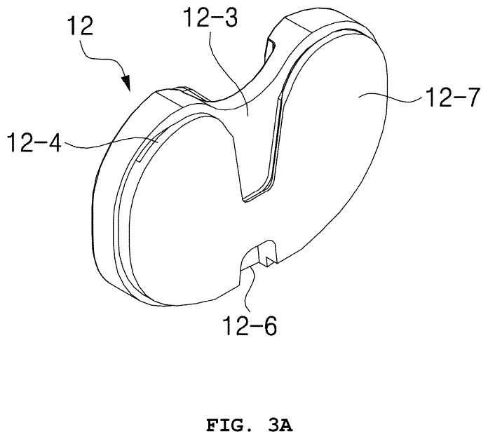

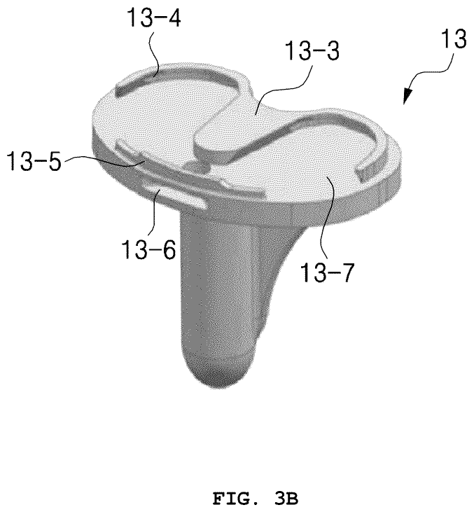

### DETAILED DESCRIPTION OF THE INVENTION

Hereinafter, preferred embodiments of the present disclosure will be described in detail with reference to the accompanying drawings.

The terminology used herein to describe the specific embodiment of the present disclosure is not intended to limit the scope of the present disclosure. The singular expression includes the plural expression unless the context clearly dictates otherwise.

In this specification, the terms “comprise”, “include”, or “have” are intended to indicate that there is a feature, number, step, action, component, part, or combination thereof described in the specification, and it is to be understood that the present disclosure does not exclude the possibility of the presence or the addition of one or more other features, numbers, steps, operations, components, parts, or combinations thereof.

It will be understood that, although the terms “first,” “second,” etc. may be used herein to describe various components, these components should not be limited by these terms. These terms are only used to distinguish one component from another.

In the description with reference to the accompanying drawings, the same reference numerals will be given to the same components, and overlapping descriptions thereof will be omitted. In describing the present disclosure, if it is determined that a detailed description of a related known technology may unnecessarily obscure the subject matter of the present disclosure, the detailed description will be omitted.

In addition, in describing the embodiments of the present disclosure, directional terms to be used later will be defined as follows.

The “first direction” is the front-rear direction of an artificial knee joint replacement according to an embodiment of the present disclosure, and the “second direction” is defined as the width direction of the artificial knee joint replacement orthogonal to the first direction. It is preferable to understand that the “front” refers to a side where the artificial knee joint replacement is adjacent to the patella in surgery to replace the knee joint with the artificial knee joint replacement according to an embodiment of the present disclosure, and means the anterior direction of the body, whereas the “rear” refers to the opposite side of the patella, and means the posterior direction of the body.

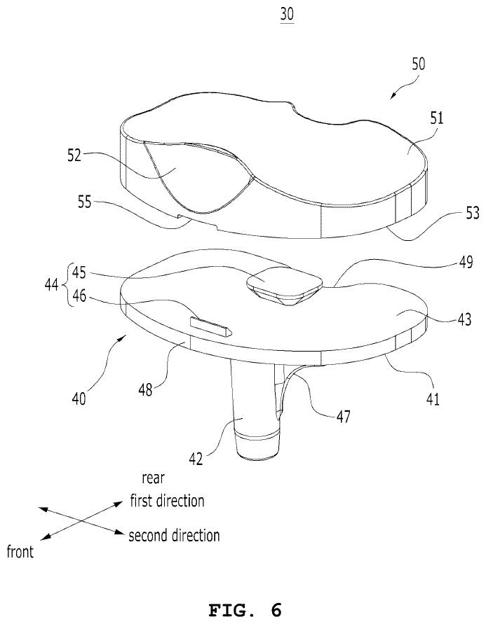

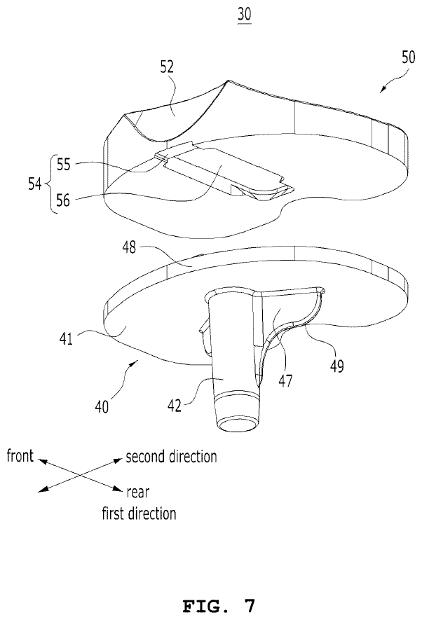

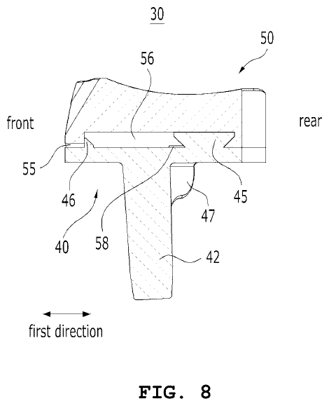

FIG. 6 is an exploded perspective view of an artificial knee joint replacement according to an embodiment of the present disclosure, FIG. 7 is a perspective view of the artificial knee joint replacement shown in FIG. 6 viewed from another angle, and FIG. 8 is a combined cross-sectional view of the artificial knee joint replacement according to an embodiment of the present disclosure.

Referring to FIGS. 6 to 8, an artificial knee joint replacement 30 according to an embodiment of the present disclosure includes a tibial component 40 and a bearing component 50. The tibial component 40 is implanted in the place where the articular surface of the upper end of the tibia adjacent to the femoral joint is resected. The bearing component 50 is positioned between the femoral component (not shown) and the tibial component 40, and serves as a kind of cartilage so that the articular surface can move smoothly.

The tibial component 40 may be firmly and directly fixed in a partial insert method using a core material 42 protruding downward at a certain height at a position where the articular surface of the upper end of the tibia is resected (bone resection surface). The tibial component 40, which is directly fixed to the bone resection surface of the tibia as such, may be composed of a lower binding surface 41 having the core material 42 in the form of a single object and an upper fastening surface 43 to which the bearing component 50 is fastened.

As mentioned above, the bearing component 50 is interposed between the femoral component directly fixed to the bone resection surface of the femur and the tibial component 40 to perform the role of cartilage. The bearing component 50 may consist of an upper support surface 51 having a pair of curved concave contact portions to which the femoral component is in direct contact bendably and a lower fastening surface 53 coupled to the tibial component 40.

The bearing component 50 may be a composite of one selected from ultra high molecular weight polyethylene (UHMWPE), polyether ether ketone (PEEK), carbon fiber reinforced polymer, and glass fiber reinforced polymer, or a combination of two or more. At this time, the UHMWPE may be conventional PE (CPE) or highly crosslinked PE (HXLPE), and in some cases, vitamin E may be further included in the UHMWPE.

For reference, vitamin E has an antioxidant action and is known to be effective in treating arthritis. Therefore, vitamin E may reduce the possibility of inflammation after total knee arthroplasty.

The tibial component 40 may include a protrusion portion 44 protruding from the upper fastening surface 43. The bearing component 50 may have a configuration in which an accommodation portion 54 is formed on the lower fastening surface 53 to correspond to the protrusion portion 44. The protrusion portion 44 may consist of two protrusions and 46 having different shapes, and the accommodation portion 54 may consist of grooves 55, 56 that accommodate the two protrusions 45 and 46 and are coupled with the corresponding protrusions at the same time.

In FIGS. 6 and 7, unexplained reference numeral 52 indicates a patella accommodation surface that accommodates a part of the patella (kneecap), and 47 indicates a rib for reinforcing the core material 42.

The configuration of the artificial knee joint replacement according to the embodiment of the present disclosure will be examined in more detail for each component.

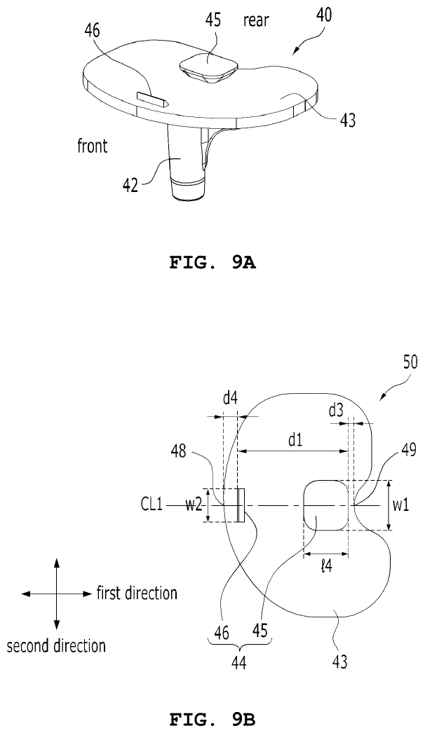

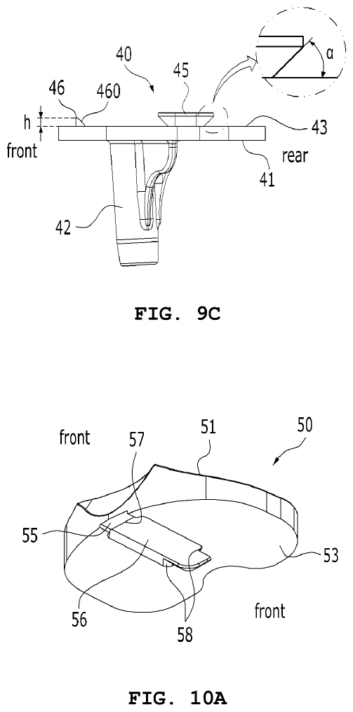

FIGS. 9A to 9C are views of the tibial component 40 applied to the artificial knee joint replacement according to an embodiment of the present disclosure. FIGS. 9A, 9B, and 9C are a perspective view, a plan view, a right side view of the tibial component, respectively. With reference to this, the configuration of the tibial component will be described first.

Referring to FIGS. 9A to 9C, the tibial component 40 may be firmly and directly fixed to the articular surface of the upper end of the tibia by a partial insert method using a core material 42 protruding downward at a certain height. The tibial component 40, which is directly fixed to the bone resection surface of the tibia as such, may be composed of the lower binding surface 41 having the core material 42 in the form of a single object and the upper fastening surface 43 to which the bearing component 50 is fastened.

The upper fastening surface 43 of the tibial element 40 may be provided with the protrusion portion 44 protruding from the upper fastening surface 43 to a specific height. The protrusion portion 44 may consist of two protrusions 45 and 46 having different shapes. The two protrusions 45 and 46 constituting the protrusion portion 44 may be arranged in a form spaced apart from each other at a distance from each other in the first direction while each center thereof is located on a first direction reference line CL1 of the tibial component 40.

It is preferable that the two protrusions 45 and 46 may consist of: the first protrusion 45 protruding from the upper fastening surface 43 separated by a first distance d3 from a rear indentation portion 49 of the tibial component 40, and having a dovetail-shaped cross-sectional structure in which the width or cross-sectional area gradually decreases as going downward; and the second protrusion 46 protruding from the upper fastening surface 43 separated by a second distance d4 from an anterior curvature point 48 of the tibial component 40, and whose surface facing the first protrusion 45 is inclined downward in the direction of the first protrusion 45.

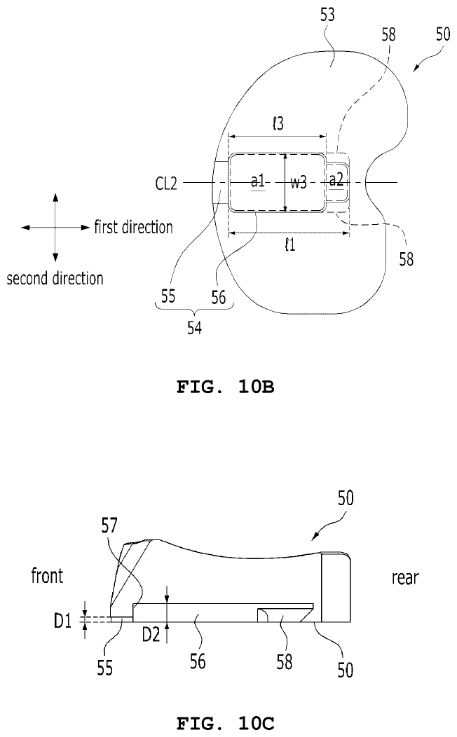

At this time, a straight line distance d1 between the outermost surface of one of the two protrusions 45 and 46 (first protrusion or second protrusion) in the first direction and the outermost surface of the other protrusion (second protrusion or first protrusion) in the first direction may be equal to a length 11 of the remaining groove except for the first groove 55 opened toward the front of the bearing component 50 among the aforementioned grooves 55 and 56 in the first direction (see FIG. 10B) or slightly greater than the length 11 in the first direction.

In addition, assuming that the second distance d4 is at least greater than the first distance d3, when applied in a patient-specific manner, it is possible to secure a relatively large processing allowance (cutting allowance) for the front surface of the tibial component 40 on a bow-shaped curved line with a relatively high processing frequency compared to the rear surface.

Due to the above configuration, when the tibial component 40 and the bearing component 50 are fastened, the outermost surface of the first protrusion 45 and the outermost surface of the second protrusion 46 are coupled to both side walls in the first direction of the other groove (the second groove 56 to be described later) in a form that is precisely engaged, or somewhat tightly coupled in the form of an interference fit, so that firm fastening or binding without any separation between each other may be achieved.

It is preferable that the first protrusion 45 has a planar uppermost surface with the largest area whose shape is a rectangle or a square as illustrated in the drawing, and is a protrusion having a dovetail-shaped cross-sectional structure in which the cross-sectional area gradually decreases as the planar shape goes downward from the uppermost surface of the quadrangular shape, that is, closer to the upper fastening surface 43. At this time, an outer surface inclination angle α (dovetail inclination angle) of the dovetail-shaped first protrusion 45 is preferably between 15 and 75 degrees.

When the dovetail inclination angle α exceeds 75 degrees, the fastening force with a binding rail 58 to be described later is not sufficient, resulting in loose fastening or the first protrusion 45 may push the binding rail 58 upward and be arbitrarily separated. When the dovetail inclination angle α is less than 15 degrees, the fastening force with the binding rail 58 increases, but a large force is required for fastening as much as the increased fastening force.

Meanwhile, the second protrusion 46 provided on the tibial component 40 may have a cross-sectional shape of a right triangle with a surface facing the first protrusion 45 having the above structure inclined in the direction of the first protrusion 45, and may be a protrusion having a smaller width w2 than the width w1 of the first protrusion 45 in a second direction (left-right width direction of the artificial knee joint replacement according to the present disclosure) orthogonal to the first direction.

Next, referring to FIGS. 10A to 10C, a bearing component applied to an embodiment of the present disclosure will be described.

FIGS. 10A to 10C are views of the bearing component applied to the artificial knee joint replacement according to an embodiment of the present disclosure. FIGS. 10A, 10B, and 10C are a bottom perspective view, a bottom view, and a cutaway view of the bearing component (50), respectively.

Referring to FIGS. 10A to 10C, the bearing component 50 is interposed between the femoral component (not shown) directly fixed to the bone resection surface of the femur and the tibial component 40 to serve as a cartilage. The bearing component 50 may include: the upper support surface 51 having the pair of curved concave contact portions to which the femoral component is in direct contact bendably; and the lower fastening surface 53 coupled to the tibial component 40.

The accommodation portion 54 may be formed on the lower fastening surface 53 to correspond to the protrusion portion 44, that is, the two protrusions 45 and 46 provided on the tibial component 40 so that the two protrusions 45 and 46 may be accommodated in and coupled to the accommodation portion 54. The accommodation portion 54 may be composed of grooves 55 and 56 coupled with the two protrusions 45 and 46 while accommodating the corresponding protrusions.

At this time, the grooves 55 and 56 may be formed in a continuous structure from one side to the other along a first direction reference line CL2 of the bearing component 50.

To be specific, the grooves 55 and 56 may consist of: the first groove 55 having a depth D1 lower than the height h of the second protrusion 46 of the tibial component 40 and opening toward the front of the bearing component 50; and the second groove 56 formed in succession to the rear of the first groove 55 deeper than the first groove 55. As the second groove 56 is formed deeper than the first groove 55 (D2>D1), a step 57 may be formed at the boundary between the first groove 55 and the second groove 56.

The area of the second groove 56 may be subdivided into a binding area a1 and a binding guide area a2. Along the edge of the binding area a1, the binding rail 58 to which a part of the outer surface of the first protrusion 45 having a cross-sectional structure of a dovetail shape is engaged and fixed may be provided. The width w3 of the binding guide area a2 in the second direction may correspond to the width w1 of the first protrusion 45 or may be slightly larger, and the length 13 of the binding guide area a2 in the first direction may be 2 to 2.5 times the length 14 of the first protrusion 45 (see FIG. 9B).

Due to the above configuration, when the tibial component 40 and the bearing component 50 are fastened, the first protrusion 45 slides toward the binding area a1 by an external force in the first direction in a state in which the first protrusion 45 is inserted into the binding guide area a2 of the second groove 56, and moves from the point of entry into the binding area a1 to the exact binding point in a state of being engaged with the binding rail 58, so that fastening between the tibial component 40 and the bearing component 50 may be made stably.

The locking process of the artificial knee joint replacement according to the embodiment of the present disclosure configured as described above will be examined in connection with the action and effect of the present disclosure.

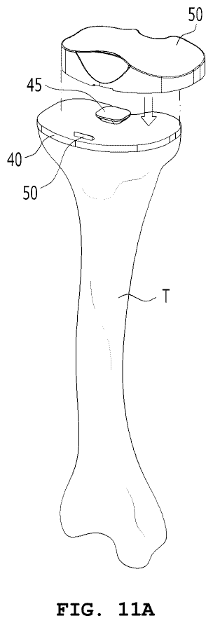

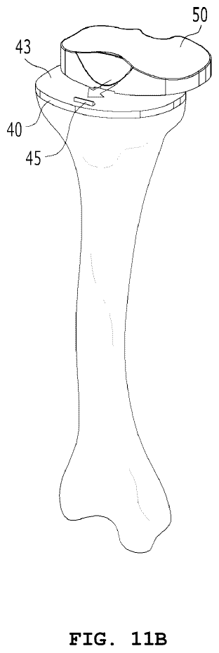

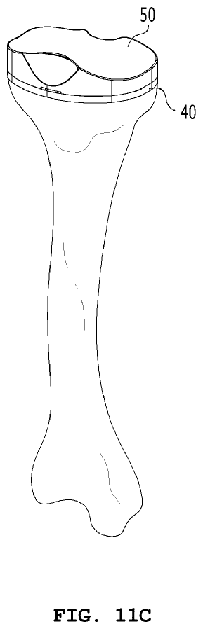

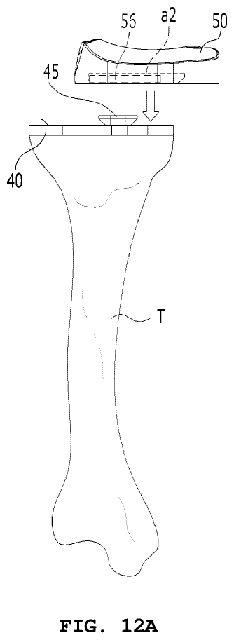

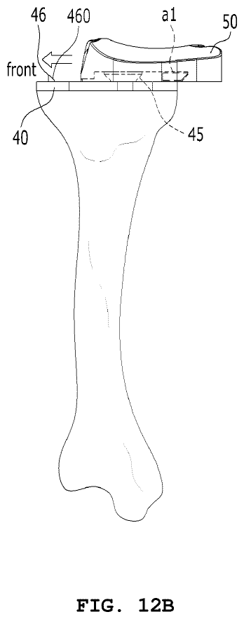

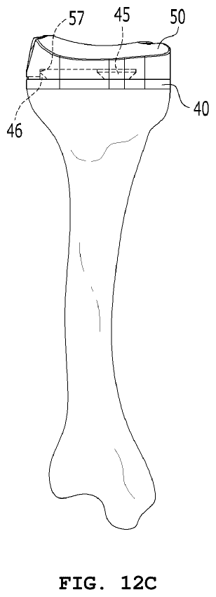

FIGS. 11A to 11C, and FIGS. 12A to 12C are views of the locking process of the artificial knee joint replacement according to an embodiment of the present disclosure. FIGS. 11A to 11C are perspective views of the locking process, and FIGS. 12A to 12C are views of the locking process viewed from the right side of the artificial knee joint replacement.

Referring to 11A to 12C, first, the bearing component 50 is positioned directly above the tibial component 40 fixed to the bone resection surface at the top of the tibia T. To be specific, the bearing component 50 is placed right above the tibial component 40 at a position where the binding guide area a2 of the second groove 56 of the bearing component 50 can engage with the first protrusion 45 of the tibial component 40 (see FIGS. 11A to 12A).

In that state (the same state as shown in FIGS. 11A to 12A), by lowering the bearing component 50 down and bringing the bearing component 50 into close contact with the upper fastening surface 43 of the tibial component 40 (see FIGS. 11A to 12B), the first protrusion of the tibial component 40 is located in the binding guide area a2 of the second groove 56, and in this state, when the bearing component 50 is pushed forward, the first protrusion 45 performs relative motion in the direction of the binding area a1 of the second groove 56 along the binding guide area a2.

Thereafter, from the point at which the first protrusion 45 enters the binding area a1, the outer surface of the first protrusion of the dovetail structure is engaged with the binding rail 58 (see FIGS. 10A to 10C) of the binding area a1 and slides toward a predetermined binding position so as not to come off arbitrarily, and a little more force is applied to the bearing component 50 at the time when a part of the second protrusion 46 of the tibial component 40 is inserted into the front opening side of the first groove 55 formed in the bearing component 50.

Then, the first groove 55 formed in the bearing component 50 moves in close contact with an inclined surface 460 of the second protrusion 46. In this process, elastic deformation occurs in which the front end of the bearing component 50 is lifted slightly upward since the height h of the second protrusion 46 is formed greater than the depth D1 of the first groove 55 (see FIGS. 9A to 10C), and the moment the first protrusion 45 reaches the predetermined binding position of the binding area a1, the front end of the bearing component 50 is elastically restored and the second protrusion 46 is caught on the step 57.

Since the straight line distance d1 (see FIG. 9B) between the outermost surfaces of the first protrusion 45 and the second protrusion 46 in the first direction is equal to the length 11 (see FIG. 10B) of the second groove 56 in the first direction or slightly greater than the length 11 in the first direction, the second protrusion 46 may be precisely engaged with the step 57 or somewhat tightly coupled in the form of an interference fit, so that firm fastening or binding between the second protrusion 46 and the step 57 may be achieved.

Meanwhile, in separating the bearing component 50 for reoperation, the bearing component 50 may be separated in the reverse order of the locking process described above. However, for separation, first, a rod-shaped separation tool is put into the opening of the first groove 55 and the front end of the bearing component 50 is lifted slightly upward using the principle of leverage to separate the second protrusion 46 from the step 57. In this state, by pulling or pushing the bearing component 50 rearward, the bearing component 50 may be separated.

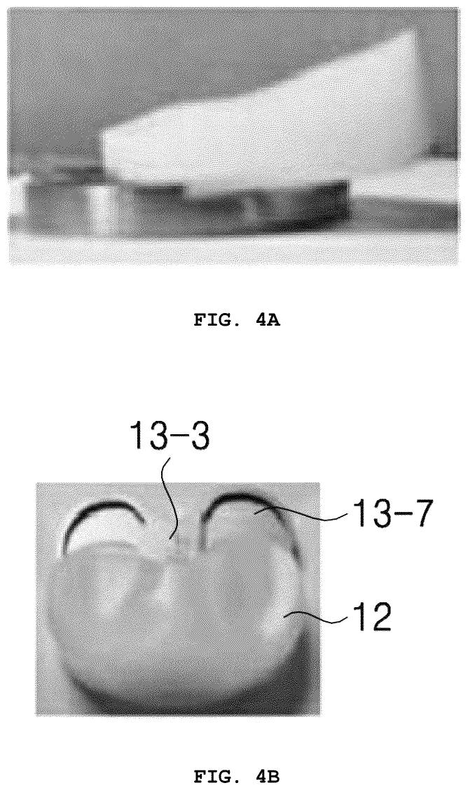

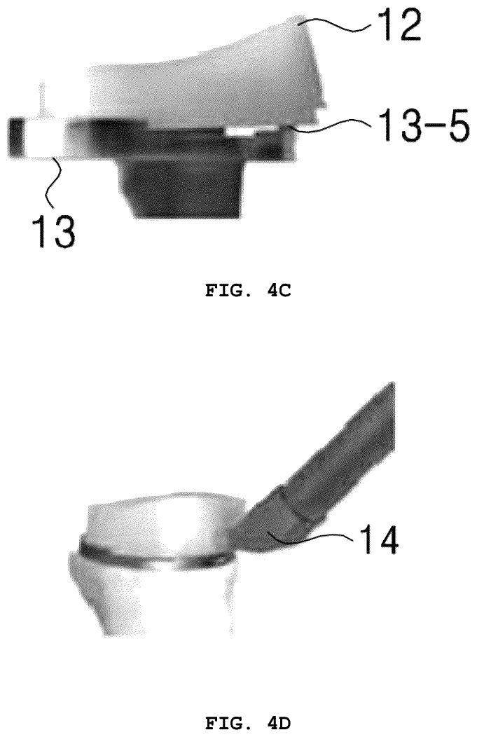

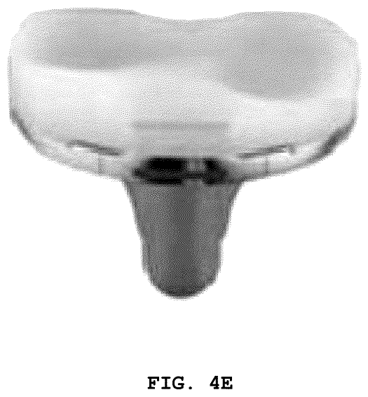

The conventional dovetail-type locking mechanism has better post-fastening stability than the hook type or pin type described above, and it is easy to separate the tibial component and the bearing component since separation is done by inserting a tool into a separation groove 12-6. Still, as shown in FIG. 4C, in the case of the dovetail type, a coupling surface 12-7 has to slide to a designated coupling position while maintaining contact with a separation preventing step 13-5 in the process of fitting a fastening portion 12-3 to an accommodation portion 13-3, which means that a lot of force is required to combine tibial component and the bearing component.

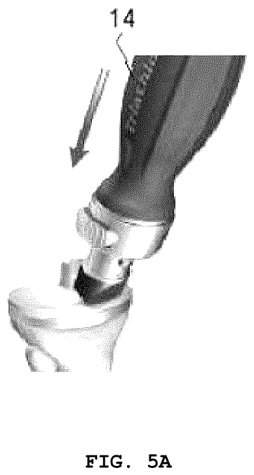

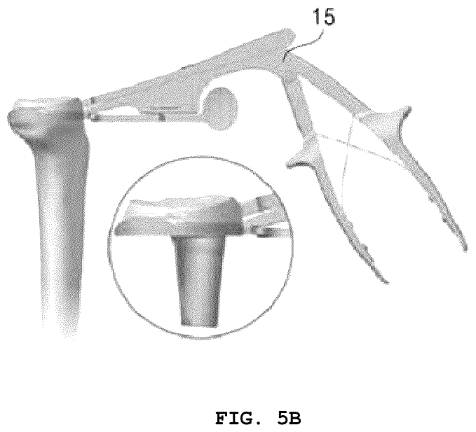

Accordingly, in an actual surgical procedure, an impactor 14 as shown in FIG. 4D or FIG. 5A or a trigger-type dedicated tool 15 or hammer as shown in FIG. 5B is used. The problem is that when an impactor or hammer is used, the impact may cause cracks in the patient's bones, and trigger-type tools require a great grip force to operate, which is inconvenient to use.

On the contrary, the artificial knee joint replacement according to an embodiment of the present disclosure adopts a dovetail locking mechanism but has a structure in which large resistance does not occur throughout the locking process unlike the case of the related art, except for a momentary resistance due to physical contact between a second protrusion and a first groove at the final stage of fastening.

That is, according to an artificial knee joint replacement according to an embodiment of the present disclosure, it is possible to compensate for the disadvantages of the conventional dovetail-type locking mechanism (excessive force required during fastening and separation) while maintaining the advantages of the dovetail-type locking mechanism (high fastening stability).

Furthermore, an artificial knee joint replacement according to an embodiment of the present disclosure has a structure that can exert strong resistance in the direction against the load applied to the knee joint of the human body during walking unlike the conventional dovetail type, in which the bearing component is locked from the front to the rear of the tibial implant, generating a large resistance in the direction opposite to the direction of the load applied to the knee joint of the actual human body during walking.

In the above detailed description of the present disclosure, only certain embodiments have been described. However, it should be understood that the present disclosure is not limited to the certain forms mentioned in the detailed description. Rather, it should be understood that the present disclosure covers all modifications, equivalents and alternatives that come within the spirit and scope of the invention as defined by the appended claims.

What is claimed is:

1. An artificial knee joint replacement, comprising:
a tibial component directly fixed to a bone resection surface of tibia and configured to have a protrusion portion protruding from an upper fastening surface thereof,
wherein the protrusion portion consists of two protrusions spaced apart from each other along a first direction reference line of the tibial component,
wherein the two protrusions comprises:
a first protrusion configured to protrude from the upper fastening surface spaced a first distance from a rear indentation portion of the tibial component, and have a dovetail-shaped cross-sectional structure in which a width or cross-sectional area gradually decreases as going downward; and
a second protrusion configured to protrude from the upper fastening surface spaced a second distance from an anterior curvature point of the tibial component, and whose surface facing the first protrusion is inclined downward in a direction of the first protrusion.

2. The artificial knee joint replacement of claim 1, wherein the first protrusion has a planar uppermost surface with a largest area whose shape is a rectangle or square, and is a protrusion having a dovetail-shaped cross-sectional structure in which a cross-sectional area gradually decreases downward.

3. The artificial knee joint replacement of claim 1, wherein the second protrusion has a cross-sectional shape of a right triangle with a surface facing the first protrusion inclined in the direction of the first protrusion, and is a protrusion having a smaller width (w2) than a width (w1) of the first protrusion in a second direction orthogonal to the first direction.

4. An artificial knee joint replacement, comprising:
a bearing component interposed between a femoral component directly fixed to a bone resection surface of femur and the tibial component described in claim 1, and configured to have an accommodation portion formed on a lower fastening surface thereof to correspond to the protrusion portion of the tibial component,
wherein the accommodation portion consists of grooves formed in a structure that is continuous with each other along a first direction reference line of the bearing component,
wherein the grooves comprises:
a first groove configured to open toward a front of the bearing component; and
a second groove formed in succession behind the first groove deeper than the first groove,
wherein the second groove is subdivided into:
a binding area provided with a binding rail; and
a binding guide area configured to guide a protrusion formed on the tibial component to slide toward the binding area to be bound.

5. An artificial knee joint replacement, comprising:
a tibial component directly fixed to a bone resection surface of tibia and configured to have a protrusion portion protruding from an upper fastening surface thereof; and
a bearing component interposed between a femoral component directly fixed to a bone resection surface of femur and the tibial component, and configured to have an accommodation portion formed on a lower fastening surface thereof to correspond to the protrusion portion,
wherein the protrusion portion consists of two protrusions spaced apart from each other along a first direction reference line of the tibial component, and
the accommodation portion consists of grooves formed in a structure that is continuous with each other along a first direction reference line of the bearing component,
wherein a straight line distance (d1) between an outermost surface of one of the two protrusions in a first direction and an outermost surface of the remaining protrusion in the first direction is equal to a length (l1) of a remaining groove except for a first groove opened toward a front of the bearing component among the grooves in the first direction or greater than the length (l1) in the first direction.

6. The artificial knee joint replacement of claim 5, wherein the two protrusions comprises:
a first protrusion configured to protrude from the upper fastening surface spaced a first distance from a rear indentation portion of the tibial component, and have a dovetail-shaped cross-sectional structure in which a width or cross-sectional area gradually decreases as going downward; and
a second protrusion configured to protrude from the upper fastening surface spaced a second distance from an anterior curvature point of the tibial component, and whose surface facing the first protrusion is inclined downward in a direction of the first protrusion.

7. The artificial knee joint replacement of claim 6, wherein the grooves comprises:
the first groove formed to a depth lesser than a height of the second protrusion; and
a second groove formed in succession behind the first groove deeper than the first groove,
wherein the second groove is subdivided into:
a binding area provided with a binding rail in which a part of an outer surface of the first protrusion having the dovetail-shaped cross-sectional structure is engaged and fixed; and
a binding guide area configured to guide the first protrusion to slide toward the binding area to be bound.

8. The artificial knee joint replacement of claim 6, wherein the first protrusion has a planar uppermost surface with a largest area whose shape is a rectangle or square, and is a protrusion having the dovetail-shaped cross-sectional structure in which a cross-sectional area gradually decreases downward.

9. The artificial knee joint replacement of claim 6, wherein the second protrusion has a cross-sectional shape of a right triangle with a surface facing the first protrusion inclined in the direction of the first protrusion, and is a protrusion having a smaller width (w2) than a width (w1) of the first protrusion in a second direction orthogonal to the first direction.

10. The artificial knee joint replacement of claim 7, wherein a moment the first protrusion moved along the binding guide area reaches a predetermined binding position in the binding area, the second protrusion is caught on a step formed at a boundary between the first groove and the second groove.

11. The artificial knee joint replacement of claim 5, wherein the bearing component is a composite of one selected from ultra high molecular weight polyethylene (UHMWPE), polyether ether ketone (PEEK), carbon fiber reinforced polymer, and glass fiber reinforced polymer, or a combination of two or more.

12. The artificial knee joint replacement of claim 11, wherein as the UHMWPE, conventional PE (CPE) or highly crosslinked PE (HXLPE) is used.

13. The artificial knee joint replacement of claim 11, wherein vitamin E is included in the UHMWPE.

14. The artificial knee joint replacement of claim 7, wherein the first protrusion has a planar uppermost surface with a largest area whose shape is a rectangle or square, and is a protrusion having the dovetail-shaped cross-sectional structure in which a cross-sectional area gradually decreases downward.

15. The artificial knee joint replacement of claim 7, wherein the second protrusion has a cross-sectional shape of a right triangle with a surface facing the first protrusion inclined in the direction of the first protrusion, and is a protrusion having a smaller width (w2) than a width (w1) of the first protrusion in a second direction orthogonal to the first direction.
